(19) United States
(12) Patent Application Publication
(10) Pub. No.: US 2025/0312141 A1
(43) Pub. Date: Oct. 9, 2025

(54) SURGICAL SYSTEMS AND METHODS INCLUDING A STAPLE FOR GRAFT FIXATION

(71) Applicant: Biomet Manufacturing, LLC, Warsaw, IN (US)

(72) Inventors: Seth Petri, Chandler, AZ (US); Ryan A. Kaiser, Leesburg, IN (US)

(21) Appl. No.: 19/087,945

(22) Filed: Mar. 24, 2025

Related U.S. Application Data
(60) Provisional application No. 63/574,785, filed on Apr. 4, 2024.

(51) Int. Cl. A61F 2/08

(52) U.S. Cl. CPC A61F 2/0811; A61F 2002/0847

(57) ABSTRACT

A system for connecting to a graft to in a reconstruction surgery, comprising: a suture construct; and a staple configured to anchor the suture construct to the graft.

### CLAIM OF PRIORITY

This application claims the benefit of U.S. Provisional Patent Application Ser. No. 63/574,785, filed on Apr. 4, 2024, the benefit of priority of which is claimed hereby, and which is incorporated by reference herein in its entirety.

### FIELD OF THE INVENTION

The present invention relates to the field of orthopedic surgery, and more specifically to surgical devices and methods including a staple for the attachment of suture constructs to tissue grafts during ligament reconstruction procedures.

### BACKGROUND

Anterior cruciate ligament (“ACL”) and other ligament injuries have become increasingly common for both athletes and non-athletes. ACL reconstruction is commonly performed to replace an injured ACL. The goal of ACL reconstruction is to restore knee stability and function. Autografts and allografts are the two primary types of grafts used in ACL reconstruction. Autografts involve the use of the patient's own tissue, while allografts use tissue from a cadaver. Autografts are often preferred due to their lower risk of rejection and better integration with the patient's body. The most commonly used tissues for autografts in ACL reconstruction are the hamstring tendon, patellar tendon, and the quadriceps tendon.

The fixation of the graft within the knee joint is a pivotal aspect of ACL reconstruction. It requires precise placement and secure attachment of the graft to the bone to allow for successful integration and healing. Various fixation methods and devices have been developed to achieve this goal, including interference screws, cross pins, suture anchors, and suspensory fixation systems (all the above are generally referred to as a suspensory fixation mechanism herein). These methods aim to provide immediate and robust fixation that can withstand the forces exerted on the knee during the healing process and return to activity.

The evolution of surgical techniques and fixation devices continues to be driven by the pursuit of improved clinical outcomes, reduced surgical time, and enhanced recovery rates for patients.

### OVERVIEW

The present application provides advances in surgical techniques, systems and fixation assemblies including the development of methods that offer strong, reliable, and easy-to-use fixation devices while minimizing the complexity of a surgical reconstruction. There is a need for more simplified and efficient surgical reconstruction, outcomes which can be improved by the present surgical techniques, systems and fixation assemblies.

The present surgical techniques, systems and fixation assemblies can simplify the process of connecting a graft to a suspensory fixation mechanism. The present application provides a “quick connect” solution that reduces the number of surgical steps required, making the technique less tedious and time consuming for the surgeon. For example, the present surgical techniques can include placing a suture construct against or adjacent the graft, affixing a quick connect (e.g., a specifically designed staple) on one or more sides of the suture construct and/or the graft, wrapping tails of the suture construct around the affixed suture construct, the staple and the graft, and then optionally securing the tails with a knot. According to one example further illustrated in FIGS. 10A and 10B, a staple, a suture construct utilizing technology such as a ZipLoop® manufactured and sold by Zimmer Biomet of Warsaw, IN, and sleeve can be configured to be passed around the graft. The suture construct can be coupled with the staple to form a closed loop. This closed loop can be passed through the staple. The closed loop and tails or another loop of the suture construct can be tightened to trap the graft as shown in FIG. 10B. This trap can couple the staple to the graft as shown in FIG. 10B and can result in the suture construct being tightly wrapped around the graft as shown in FIG. 10B. These techniques are less complex than traditional methods that typically require multiple needle piercings and more intricate wrapping. The surgical techniques, systems and fixation assemblies disclosed can reduce the total surgery time. This efficiency is beneficial for both the surgical team and the patient.

The present inventors contemplate the surgical techniques, systems and fixation assemblies disclosed herein can provide a simplified and repeatable technique for securing a suture construct to the graft. This consistency is important for ensuring successful outcomes across different surgeries and different surgeons. Additionally, the present inventors realize that the surgical techniques, systems and fixation assemblies disclosed herein provide for various additional benefits including enhanced precision, enhanced fixation (e.g., increased number of fixation points), compatibility with different grafts and graft sizes, compatibility with various configurations of suture constructs, reduced complexity, possible elimination of need for use of a needle in anchoring the suture construct to the graft and improved ease of use.

Regarding the enhanced precision facilitated by the surgical techniques, systems and fixation assemblies disclosed herein, the present devices provide for defined sizes and location of features such as grooves and/or holes allowing the surgeon to couple and/or pass sutures such as into the graft in a predefined and more precise manner. For example, by defining the locations for needle piercing with the holes in the staple, the process becomes more uniform and easier for the surgeon. Traditional methods are not so precise and instead rely on surgeon judgment and skill in affixing a suture construct to the graft. This judgment and location of fixation can vary from surgery to surgery and surgeon to surgeon.

Regarding the enhanced fixation provided by the surgical techniques, systems and fixation assemblies disclosed herein, the use of a staple to anchor the suture construct to the graft provides better fixation compared to traditional methods. The staple can offer multiple points of fixation via multiple legs, which may not be achievable with needle and suture alone.

The above described benefits of the surgical techniques, systems and fixation assemblies are provided for exemplary purposes and additional benefits not discussed herein are contemplated or may yet be realized.

The following, non-limiting examples, detail certain aspects of the present subject matter to solve the challenges and provide the benefits discussed herein, among others.
Example 1 is a system for connecting to a graft to in a reconstruction surgery, comprising: a suture construct; and a staple configured to anchor the suture construct to the graft.
In Example 2, the subject matter of Example 1 includes, wherein the suture construct includes a main portion, a link portion and a plurality of tails connected to and extending from the link portion, wherein the staple is configured to anchor the link portion to the graft.
In Example 3, the subject matter of Example 2 includes, wherein the suture construct includes a bridge with one or more features configured to receive portions of the plurality of tails.
In Example 4, the subject matter of Examples 2-3 includes, wherein the staple includes one or more grooves configured to receive one or more of the plurality of tails therein.
In Example 5, the subject matter of Example 4 includes, wherein the one or more grooves are spaced longitudinally along a longitudinal length of the staple to receive the plurality of tails which are wrapped around the graft to form a cerclage.
In Example 6, the subject matter of Examples 2-5 includes, wherein the staple includes one or more holes configured to receive one or more of the plurality of tails therein.
In Example 7, the subject matter of Example 6 includes, wherein the one or more holes are spaced longitudinally along a longitudinal length of the staple and the one or more holes are configured to receive one or more of the plurality of tails therein.
In Example 8, the subject matter of Examples 6-7 includes, a needle coupled to a first end of the plurality of tails, wherein the first end opposes a second end that is coupled to or forms the link portion, wherein the one or more holes are each configured as a guide to pass the needle through the staple and into the link portion and the graft.
In Example 9, the subject matter of Examples 2-8 includes, a card configured to carry the main portion of the suture construct.
In Example 10, the subject matter of Examples 1-9 includes, an inserter configured to engage the staple and clamp the staple against the graft to cause penetration of the staple into the suture construct and the graft.
In Example 11, the subject matter of Examples 1-10 includes, wherein the suture construct is configured to engage both a first side and a second side of the graft, wherein the staple is configured to anchor the suture construct to the first side of the graft, and further comprising a second staple configured to anchor the suture construct to the second side of the graft.
Example 12 is a method of connecting a suture construct to a graft in a reconstruction surgery, comprising: harvesting the graft; placing the suture construct adjacent the graft; and anchoring the suture construct to the graft with a staple.
In Example 13, the subject matter of Example 12 includes, anchoring the suture construct to a second side of the graft with a second staple.
In Example 14, the subject matter of Examples 12-13 includes, wrapping the suture construct around the graft and staple to form a cerclage.
In Example 15, the subject matter of Example 14 includes, passing the suture construct through one or more holes of the staple and piercing the suture construct captured by the staple and piercing the graft.
In Example 16, the subject matter of Examples 14-15 includes, passing the suture construct through one or more grooves of the staple during the wrapping.
In Example 17, the subject matter of Examples 12-16 includes, wherein anchoring the suture construct to the graft with the staple includes clamping the staple against the graft to cause penetration of the staple into the suture construct and the graft.
Example 18 is a method of connecting a suture construct to a graft in a reconstruction surgery, comprising: placing the suture construct adjacent the graft; anchoring the suture construct to the graft with a staple; and wrapping the suture construct around the graft and staple to form a cerclage.
In Example 19, the subject matter of Example 18 includes, passing the suture construct through one or more holes of the staple and piercing the suture construct captured by the staple and piercing the graft during the wrapping.
In Example 20, the subject matter of Examples 18-19 includes, passing the suture construct through one or more grooves of the staple during the wrapping.
Example 21 is an assembly for a reconstruction surgery, comprising: a suture construct including a main portion, a link portion and a plurality of tails connected to and extending from the link portion; and a staple coupled to the link portion and configured to anchor the link portion to a graft, wherein when the link portion is anchored to the graft by the staple, the plurality of tails are configured to be wrapped around the graft and the staple to form a cerclage.
In Example 22, the subject matter of Example 21 includes, wherein the suture construct includes a bridge with one or more features comprising at least one of a groove and a hole configured to receive portions of the plurality of tails.
In Example 23, the subject matter of Example 22 includes, a needle coupled to a first end of the plurality of tails, wherein the first end opposes a second end that is coupled to or forms the link portion, wherein the hole is configured as a guide to pass the needle through the staple and into the link portion and the graft.
In Example 24, the subject matter of Examples 21-23 includes, a card carrying the main portion of the suture construct.
In Example 25, the subject matter of Examples 21-24 includes, an inserter configured to engage the staple and clamp the staple against the graft to cause penetration of the staple into the link portion and the graft.
In Example 26, the subject matter of Examples 21-25 includes, wherein the assembly includes the anchor and a second anchor anchoring the suture construct to both a first side and a second side of the graft, respectively.
Example 27 is an apparatus comprising means to implement of any of Examples 1-26.
Example 28 is a system to implement of any of Examples 1-26.
Example 29 is a method to implement of any of Examples 1-26.
In Example 30, the assemblies, instruments, methods or systems of any one or any combination of Examples 1-26 can optionally be configured such that all elements or options recited are available to use or select from.

### BRIEF DESCRIPTION OF THE DRAWINGS

Various embodiments of the present invention will now be discussed with reference to the appended drawings. It should be appreciated that the drawings depict only typical embodiments of the invention and are therefore not to be considered limiting in scope.

FIG. 1 is a schematic view of some components of a surgical assembly including a suture construct, a card and a needle used during a ligament reconstruction procedure according to an example of the present disclosure.

FIG. 2 is a schematic view of some components of another surgical assembly including a suture construct and a card used during a ligament reconstruction procedure according to an example of the present disclosure.

FIG. 3 is a perspective view of a staple that can be used as part of the surgical assembly of either FIG. 1 or 2 to anchor the suture construct to a graft according to an example of the present disclosure.

FIG. 4 is a perspective view of another staple that can be used as part of the surgical assembly of either FIG. 1 or 2 to anchor the suture construct to a graft according to an example of the present disclosure.

FIGS. 5A-5E illustrate a method of connecting the suture construct to a graft using the staple of FIG. 3 or 4 during a ligament reconstruction procedure according to an example of the present disclosure.

FIG. 6 is a plan view of portions of an inserter engaging with a staple such as the staple of FIG. 3 or 4 according to an example of the present disclosure.

FIGS. 7A-7C are plan views of another inserter engaging with a staple such as the staple of FIG. 3 or 4 according to an example of the present disclosure.

FIG. 8 is a highly schematic view of an assembly and a graft with portions of two suture constructs anchored to the graft by a plurality of staples according to an example of the present disclosure.

FIG. 9 is a highly schematic view of an assembly and a graft with portions of two suture constructs anchored to the graft by a plurality of staples according to an example of the present disclosure.

FIG. 10A is perspective view of an assembly including a sleeve, a staple and a suture construct, the sleeve being configured to receive the graft of the ligament therein according to an example of the present disclosure.

FIG. 10B is a perspective view of the staple and the suture construct secured to the graft of the ligament upon removal of the sleeve of FIG. 10A according to an example of the present disclosure.

### DETAILED DESCRIPTION OF THE INVENTION

The present invention pertains to surgical techniques, systems and fixation assemblies designed to facilitate the attachment of tissue grafts to suspensory fixation mechanisms during reconstructive procedures, such as ACL reconstruction surgery. Examples of the present invention include a suture construct, equipped with a main portion, a link portion, and multiple tails, which is anchored to the graft using a specifically designed staple(s). The staple, which can be inserted using an inserter tool, is configured to secure the suture construct to the graft, allowing the tails to be wrapped around the graft, portions of the suture construct and staple, forming a cerclage. The surgical techniques, systems and fixation assemblies streamline the graft attachment process, offering the benefits described previously. Although described in reference to using a quadriceps tendon, the surgical techniques, systems and fixation assemblies discussed herein can be used with various other tendon(s) from other joints. Thus, the surgical techniques, systems and fixation assemblies are not limited to the quadriceps tendon or to ACL reconstruction surgery. Several embodiments of the surgical techniques, systems and fixation assemblies will now be described to provide an overall understanding of the principles of the form, function and methods of use. This description of the general principles of this invention is not meant to limit the inventive concepts in the appended claims.

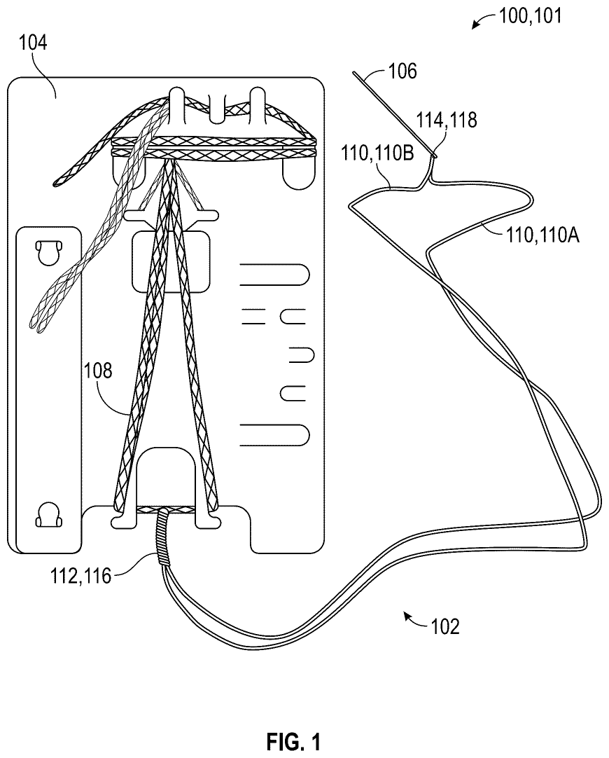

FIG. 1 illustrates an assembly 100 and system 101 including a suture construct 102, a card 104 and a needle 106 according to one example. The suture construct 102 can include a main portion 108, a plurality of tails 110, a first link portion 112 and a second link portion 114.

The suture construct 102, particularly the main portion 108, can be at least partially captured and carried by the card 104 as shown in FIG. 1. Card 104 can maintain the main portion 108 from entanglement with itself and the plurality of tails 110, for example. The plurality of tails 110 can be smaller diameter suture(s) connected to the main portion 108 such as by braiding, piercing, connection component or other known methods or components. The first link portion 112 can be located at or adjacent the connection with the main portion 108, for example. The first link portion 112 can be created by braiding or interweaving two or more of the plurality of tails 110 (e.g., the tail 110A with the tail 110B) at a first end 116, for example. However, according to other examples the first link portion 112 can be formed by a dedicated component (e.g., a suture sleeve, kite, button, intermediate component, further suture, etc.) that is separate from the plurality of tails 110.

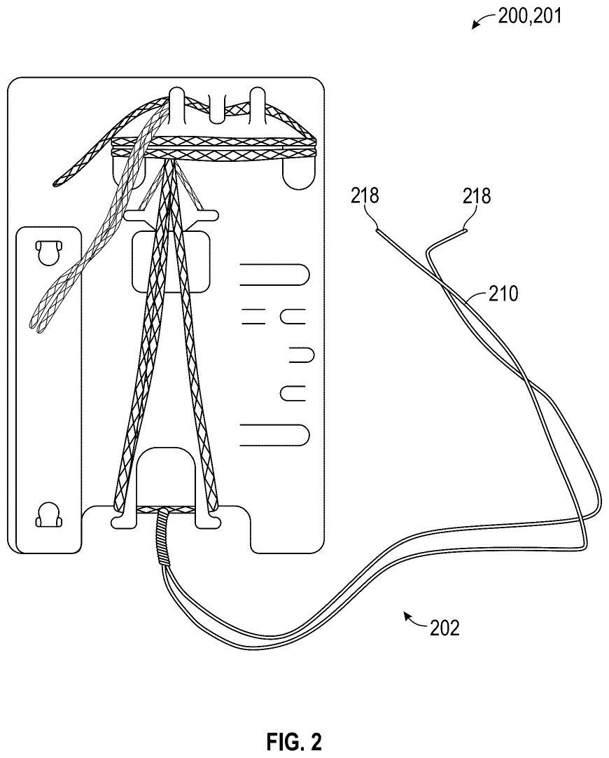

The plurality of tails 110 can extend from the first end 116 to a second end 118. The example of FIG. 1 illustrates the plurality of tails 110 configured as a continuous loop with the second link portion 114 at the second end 118. However, other examples such as that of FIG. 2 contemplate the plurality of tails being free of other features such as being open ended (e.g., not continuous loop). The needle 106 can be coupled to the second end 118, in particular, can be coupled at the second link portion 114. Although the needle 106 is illustrated in the example of FIG. 1, other examples of the assembly and methods described herein may not utilize the needle 106.

The suture construct 102 can of any type and number of sutures. Thus, sutures of different sizes, types, colors and shapes are contemplated. For example, the main portion 108 can be of one type and/or size while the plurality of tails 110 can be of another type and/or size. Any type of suture as known in the art, (e.g., broadband, ribbon, round, mesh, braided, monofilament, metal, polymer, etc.) can be utilized (e.g., braided tape, etc.) for the suture construct 102.

According to the example of FIG. 1, the plurality of tails 110 can be configured as suture tape having a lateral dimension of 1.5 mm and an approximate length of about 20 inches (e.g., 508 mm). However, these dimensions are merely exemplary and other dimensions for the plurality of tails 110 are contemplated. The needle 106 can be straight, having a diameter of around 1 mm or less. The needle 106 can be pointed, spatulated, reverse cutting, tapercut, straight cutting, conventional cutting, quill barbed, etc. Although illustrated as straight in the example of FIG. 1, the needle 106 can be curved or have another shape (quarter circle, half circle, ⅜ circle, ⅝ circle, j-shape, compound curved, etc.). The proximal end of the needle 106 can have a sharp. The card 104 can be formed of cardboard, plastic or other suitably rigid material that can be provided with suture capturing features.

FIG. 2 illustrates an assembly 200 and system 201 of similar construction to that of the assembly 100 described previously. However, the assembly 200 differs from the assembly 100 in that the assembly 200 utilizes a suture construct 202 with a plurality of tails 210 with open/free second ends 218. Thus, the plurality of tails 210 do not form a continuous loop as with the assembly 100 of FIG. 1. Furthermore, no needle need be utilized with the assembly 200. Thus, the present application anticipates that at least some of the assemblies disclosed including the staples discussed subsequently can eliminate the need for use of a needle to fixate the suture construct to the graft.

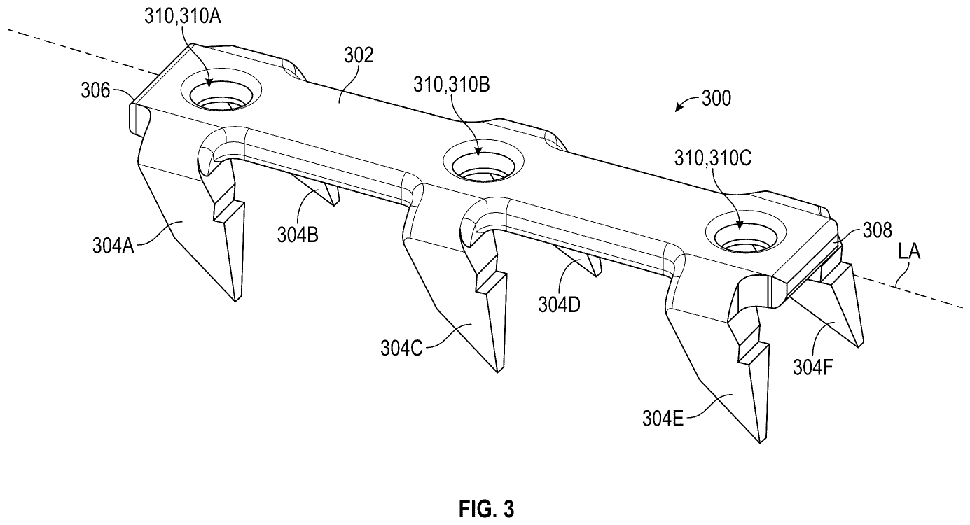

FIG. 3 shows a staple 300 according to one example. The staple 300 can be part of the assembly 100 or 200 and system 101 or 201 (FIGS. 1 and 2) discussed previously. The staple 300 can include a main body 302 and legs 304A, 304B, 304C, 304D, 304E and 304F. The main body 302 can have an elongate length, a first longitudinal end 306, a second longitudinal end 308, a longitudinal axis LA and a plurality of holes 310A, 310B and 310C.

The staple 300 can be appropriately sized given the size of the graft and the size of the suture construct being fixated to the graft. The staple 300 can be formed of suitable biostable materials such as metal, metal alloy, rigid plastic (e.g., polyetheretherketone (PEEK)), etc. The staple 300 can be bi-symmetrical in configuration. However, other shapes and symmetry are contemplated.

The legs 304A, 304B, 304C, 304D, 304E and 304F couple with lateral sides of the main body 302 and are spaced in pairs along the elongate length of the main body 302. Put another way, the legs 304A and 304B are arranged on opposing sides of the main body 302 from one another adjacent the first longitudinal end 306. The legs 304C and 304D are arranged on opposing sides of the main body 302 generally positioned at a middle of the elongate length. The legs 304E and 304F are arranged on opposing sides of the main body 302 adjacent the second longitudinal end 308. The legs 304A, 304B, 304C, 304D, 304E and 304F can be shaped for penetrating the suture construct (shown previously) and graft for fixation. Thus, the legs 304A, 304B, 304C, 304D, 304E and 304F can have sharps (e.g., points, bladed edges, barbs, etc.) as known in the art. The legs 304A, 304B, 304C, 304D, 304E and 304F can be angled as desired (e.g., canted to have points/ends further adjacent the second longitudinal end 308 than the respective connecting portions with the main body 302). The arrangement of the legs 304A, 304B, 304C, 304D, 304E and 304F shown in FIG. 3 is purely exemplary and other arrangements (e.g., non-paired and slinkgered, etc.), shape, size and number are contemplated.

The plurality of holes 310A, 310B and 310C can be formed by and can extend through the main body 302. The plurality of holes 310A, 310B and 310C can be spaced from one another along the elongate length in a predetermined arrangement having a predetermined spacing. For example, the hole 310A can be adjacent the legs 304A and 304B and the first longitudinal end 306. The hole 310B can be adjacent the legs 304C and 304D and are generally positioned at a middle of the elongate length. The hole 310C can be adjacent the legs 304E and 304F adjacent the second longitudinal end 308. The plurality of holes 310A, 310B and 310C can be appropriately sized (e.g., having a desired diameter such as between 0.5 mm and 3 mm) in order to receive and allow for passage through of the needle (e.g., the needle 106 of FIG. 1) and the tails (e.g., the plurality of tails 110 or 210 of FIG. 1 or 2). Put another way, the plurality of holes 310A, 310B, and 310C can each be configured as a guide to pass the needle (e.g., the needle 106 of FIG. 1) through the staple 300 and into underlying components such as the link portion (e.g., the first link portion 112 of FIG. 1) and the graft as further discussed and illustrated herein. The arrangement of the plurality of holes 310A, 310B and 310C shown in FIG. 3 is purely exemplary and other arrangements (e.g., non-paired with legs, non-symmetrically spaced, etc.), size and number are contemplated.

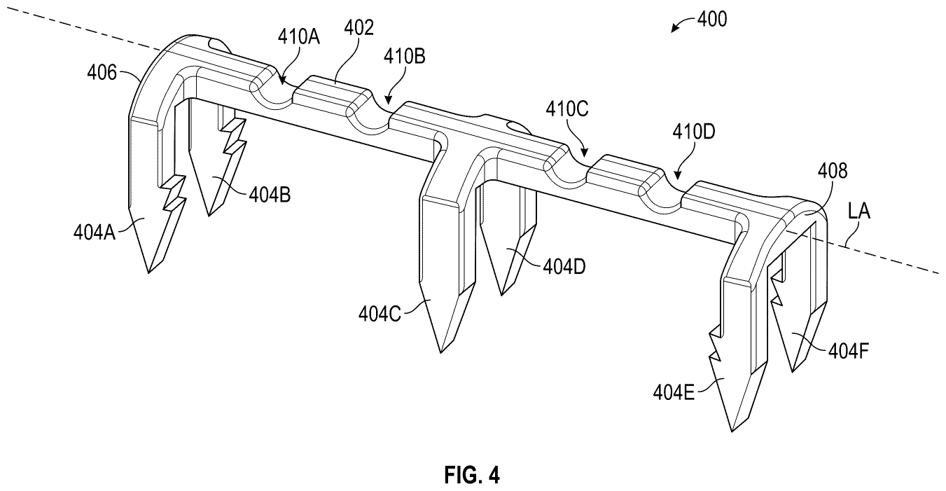

FIG. 4 shows a staple 400 according to another example. The staple 400 can be part of the assembly 100 or 200 and system 101 or 201 (FIGS. 1 and 2) discussed previously. The staple 400 can be constructed in a manner similar to that of the staple 300 described previously. Thus, the staple 400 can include a main body 402 and legs 404A, 404B, 404C, 404D, 404E and 404F. The main body 402 can have an elongate length, a first longitudinal end 406, a second longitudinal end 408, a longitudinal axis LA. The staple 400 differs in shape from the staple 300 (FIG. 3) including a thicker main body 402 and includes a plurality of grooves 410A, 410B, 410C and 410D in the main body 402 rather than the plurality of holes 310 (FIG. 3) described previously.

The legs 404A, 404B, 404C, 404D, 404E and 404F differ from the legs 304A, 304B, 304C, 304D, 304E and 304F of FIG. 3 in that the legs 404A, 404B, 404C, 404D, 404E and 404F are oriented at substantially 90 degrees to the main body 402 and the longitudinal axis LA. Thus, the legs 404A, 404B, 404C, 404D, 404E and 404F extend straight from the main body 402 and are not angled toward a first or second longitudinal end in the manner of the legs 304A, 304B, 304C, 304D, 304E and 304F of FIG. 3.

The plurality of grooves 410A, 410B, 410C and 410D can extend generally laterally across the main body 402. The plurality of grooves 410A, 410B, 410C and 410D can be spaced from one another along the elongate length in a predetermined arrangement having a predetermined spacing. For example, the grooves 410A and 410B can be spaced between the legs 404A and 404B and the legs 404C and 404D. The grooves 410C and 410D can be spaced between the legs 404C and 404D and the legs 404E and 404F. The plurality of grooves 410A, 410B, 410C and 410D can be appropriately sized (e.g., having a desired diameter or depth such as between 0.5 mm and 3 mm) in order to receive and allow for passage through of the tails (e.g., the plurality of tails 110 or 210 of FIG. 1 or 2) as further shown and illustrated herein.

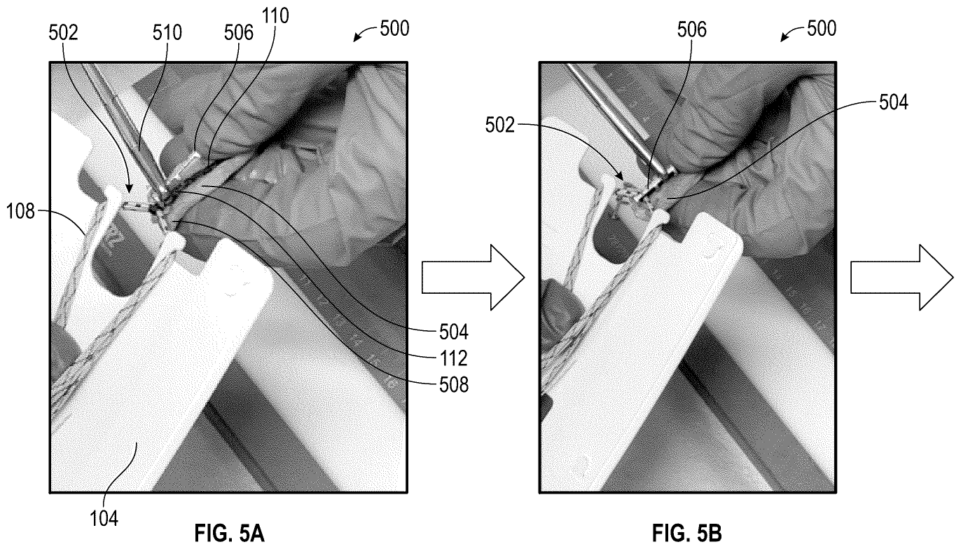

FIGS. 5A-5E illustrate a method 500 of connecting a suture construct 502 (e.g., the suture construct 102 of FIG. 1 or the suture construct 202 of FIG. 2) to a graft 504 using a staple 506 (e.g., staple 300 of FIG. 3 or staple 400 of FIG. 4) during a during a ligament reconstruction procedure such as an ACL reconstruction.

The graft 504 can be harvested from a quadriceps tendon, for example. The suture construct 502 can be part of the assemblies and systems discussed previously and can include the card 104, the main portion 108, the plurality of tails 110 and the first link portion 112 as shown in FIG. 5A.

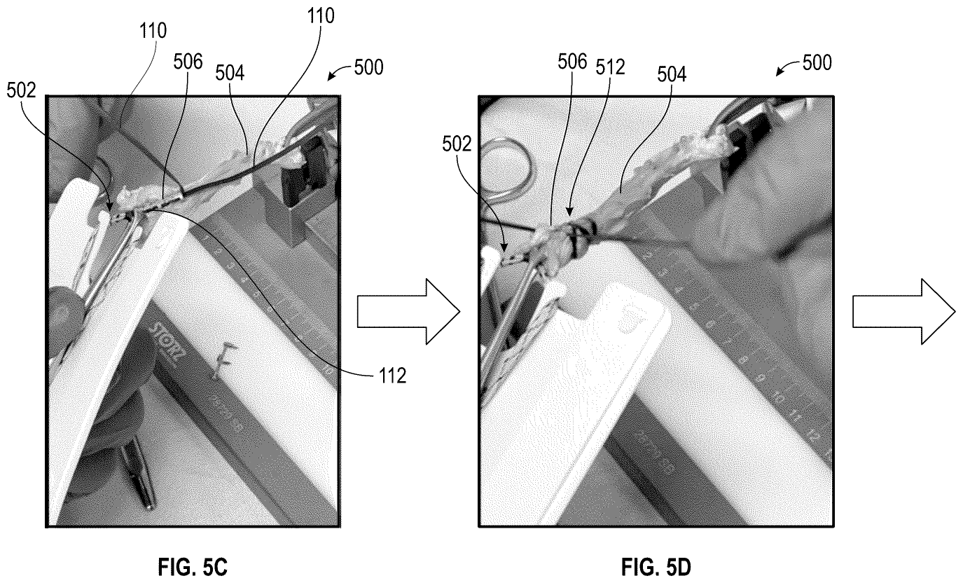

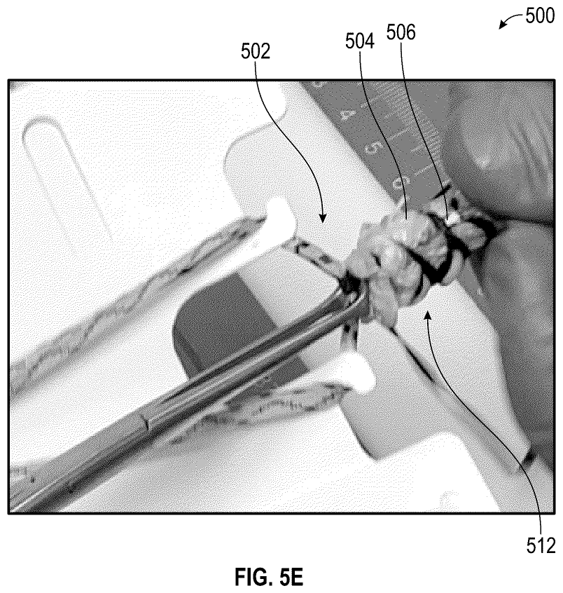

FIG. 5A shows the first link portion 112 placed adjacent or against the graft 504 such as at or adjacent a first end 508 thereof. The first link portion 112 and the plurality of tails 110 can be generally aligned along a longitudinal length of the graft 504, for example. As shown in FIGS. 5A and 5B, the surgeon positions the staple 506 relative to the first link portion 112 and the graft 504 such that the staple 506 is generally co-aligned with the first link portion 112. The staple 506 can be sized and shaped to have the legs thereof penetrate the first link portion 112 when the staple 506 is implanted down into the graft 504. The staple 506 can be positioned and implanted using an inserter 510 as illustrated. Further examples of inserters are illustrated subsequently in reference to FIGS. 6 and 7A-7C.

FIG. 5C shows the staple 506 implanted (penetrating the first link portion 112 and the graft 504) to couple/anchor the suture construct 502 to the graft 504. FIG. 5C additionally shows the plurality of tails 110 being separated after implantation of the staple 506. Turning to FIG. 5D, the plurality of tails 110 are in the process of being wrapped around the graft 504 to form a cerclage 512. FIG. 5E illustrates the cerclage 512 as completed with one or more of the plurality of grooves of the staple 506 receiving the plurality of tails 110 which are wrapped around the graft 504 and the first link portion (not shown in FIG. 5E) to form the cerclage 512.

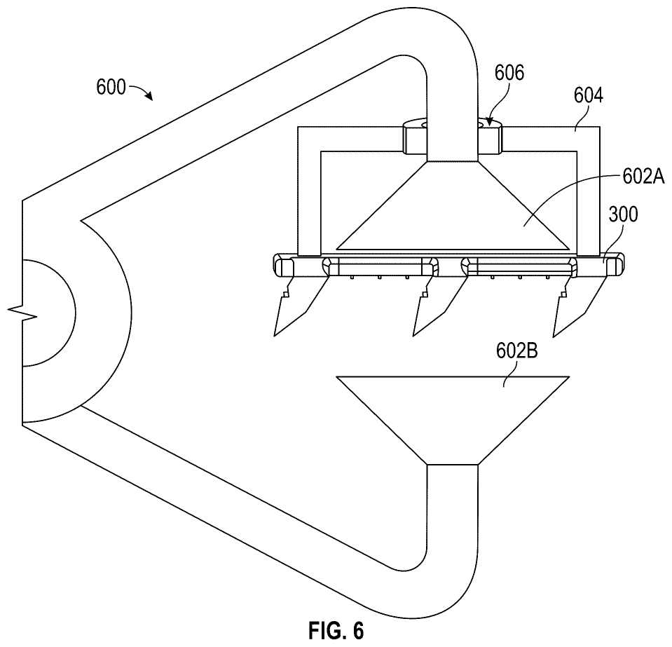

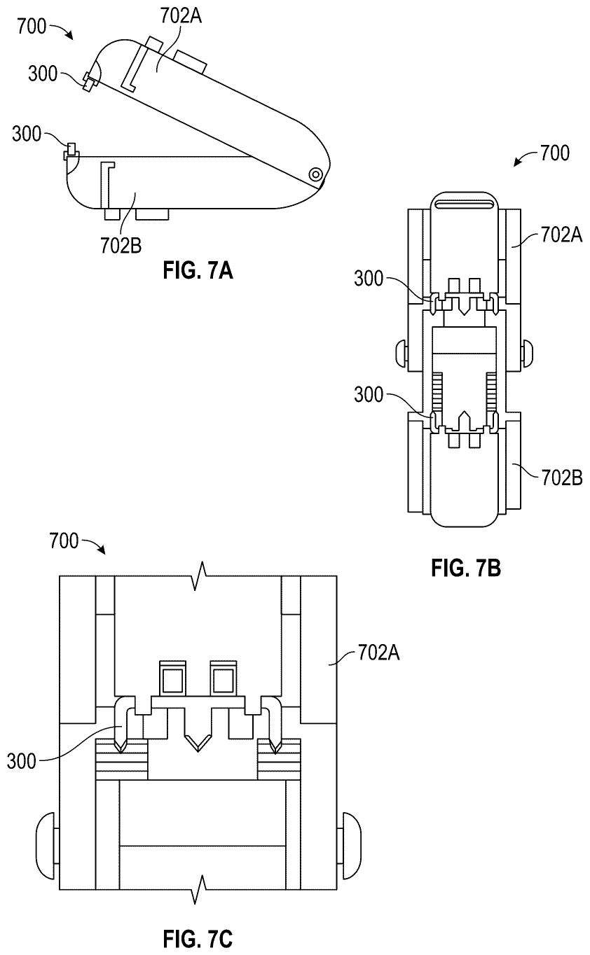

The method 500 can include harvesting the graft 504 and placing the suture construct 502 adjacent the graft 504 as shown in FIGS. 5A and 5B, for example. The method 500 can include anchoring the suture construct 502 to the graft 504 with the staple 506 as shown in FIG. 5C. The method 500 can include coupling the suture construct 502 to a suspensory fixation mechanism. Optionally, the method 500 can include anchoring the suture construct to a second side of the graft 504 with a second staple as further illustrated in reference to FIGS. 8 and 9, for example. The method 500 can include wrapping the suture construct around the graft 504 and staple 506 to form the cerclage 512 as shown in FIGS. 5D and 5E. The method 500 can optionally include passing the suture construct through one or more holes of the staple and piercing the suture construct captured by the staple and piercing the graft. The method 500 optionally can include passing the suture construct through one or more grooves of the staple during the wrapping. Anchoring the suture construct to the graft with the staple includes clamping the staple against the graft to cause penetration of the staple into the suture construct and the graft.

FIG. 6 shows a portion of an example inserter 600 engaging the staple 300 as would be done during implantation of the staple 300 into the graft and the suture construct as previously illustrated and described. The inserter 600 can be configured as a pliers having opposing moveable jaws 602A and 602B. At least the jaws 602A can be configured to engage the staple 300 along the main body thereof. Pressure from the jaws 602A can cause the implantation of the staple 300 into the graft and the suture construct. The second of the jaws 602B can engage an opposing side of the graft (not shown), for example. Optionally, a clamp attachment 604 can be used with the inserter 600 to stabilized the jaws 602A and guide them in engagement with the staple 300. The clamp attachment 604 can be configured to insert into some of the plurality of holes in the staple 300 and includes a guiding aperture 606 configured to receive a portion of the inserter 600 adjacent the jaws 602A. The clamp attachment 604 can be configured to keep the inserter 600, in particular the jaws 602A aligned with the main body of the staple 300.

FIGS. 7A-7C show another example of an inserter 700 having opposing moveable jaws 702A and 702B. The inserter 700 can be configured to capture and selectively hold two staples 300 therein, one on each of the opposing jaws 702A and 702B. This configuration can allow the staples 300 to be implanted simultaneously on opposing first and second sides of the graft (not shown). Optionally, one of the staples 300 need not be loaded onto the jaws 702A or 702B if only a single staple 300 is desired to be implanted.

FIG. 8 shows a highly schematic representation of an assembly 800 coupled to a graft 804. The assembly 800 can include a suture construct 802, a first staple 806, a second staple 808 and a second suture construct 810. The suture construct 802 can include the first link portion 112 and the plurality of tails (not shown) configured in the manner previously discussed. The first staple 806 can be implanted through the first link portion 112 on a first side of the graft 804 such as adjacent a first longitudinal end of the graft 804. As shown in FIG. 8, the first staple 806, when implanted, penetrates the first link portion 112 and the graft 804 thereby anchoring the suture construct 802 into the graft 804. The second staple 808 can be implanted through another link portion 112A of the second suture construct 810 on a second side of the graft 804 such as adjacent a second longitudinal end of the graft 804. As shown in FIG. 8, the second staple 808, when implanted, penetrates the another link portion 112A and the graft 804 thereby further anchoring the second suture construct 810 into the graft 804.

It should be noted that the example of FIG. 8 does not illustrate the tails or the cerclage of the graft 804 and/or the staples 806, 808 as discussed previously with the understanding that this can be performed if desired. Additionally, FIG. 8 does not illustrate the plurality of tails penetrating the first link portion 112, the another link portion 112A and the graft 804 as can optionally be performed with passage of a needle and the tails through hole(s) of the first staple 806 and/or the second staple 808 as discussed previously with the understanding that this can be performed if desired.

FIG. 9 shows a highly schematic representation of an assembly 900 coupled to the graft 804. The assembly 900 can include a suture construct 902, a first staple 906, a second staple 908, a third staple 910, a fourth staple 912 and a second suture construct 914. The suture construct 902 can include the first link portion 112 and the plurality of tails (not shown) configured in the manner previously discussed. However, the suture construct 902 differs from the suture construct 802 of FIG. 8 in that the first link portion 112 can be configured to cover opposing sides of the graft 804.

The first staple 906 can be implanted through the first link portion 112 on a first side of the graft 804 such as adjacent a first longitudinal end of the graft 804. The second staple 908 can be implanted through link the first link portion 112 on a second opposing side of the graft 804. As shown in FIG. 9, the first staple 906 and the second staple 908 when implanted penetrates the first link portion 112 and the graft 804 thereby anchoring the suture construct 902 into the graft 804. The third staple 910 can be implanted through the another link portion 112A on a second side of the graft 804 such as adjacent a second longitudinal end of the graft 804. The fourth staple 912 can be implanted through the another link portion 112A on a second opposing side of the graft 804 from the third staple 910. As shown in FIG. 9, the third staple 910 and the fourth staple 912, when implanted, penetrates the another link portion 112A and the graft 804 thereby further anchoring the second suture construct 914 into the graft 804.

It should be noted that the example of FIG. 9 does not illustrate the tails or the cerclage of the graft 804 and/or the staples 906, 908, 910 and 912 as discussed previously with the understanding that this can be performed if desired. Additionally, FIG. 9 does not illustrate the plurality of tails penetrating the first link portion 112, the another link portion 112A and the graft 804 as can optionally be performed with passage of a needle and the tails through hole(s) of the staples 906, 908, 910 and 912 as discussed previously with the understanding that this can be performed if desired.

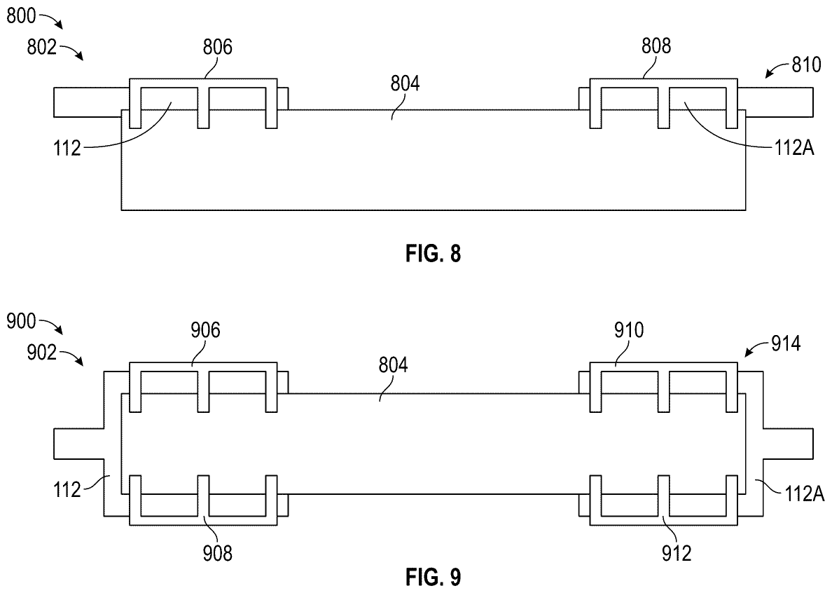

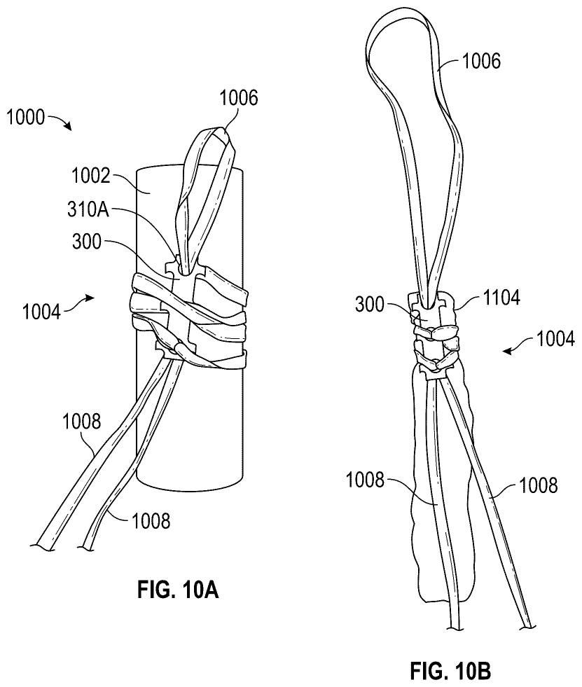

FIG. 10A shows an assembly 1000 including the staple 300 as previously described, a sleeve 1002 and a suture construct 1004. The staple 300 can be coupled to the suture construct 1004 and the suture construct 1004 can be wrapped around the sleeve 1002 as shown in FIG. 10A. Additionally, the suture construct 1004 can be passed under the staple 300 in the space between the body of the staple 300 and the sleeve 1002 and can be passed through at least one of the plurality of holes 310A to form a closed loop 1006 on a first side and tails 1008 or a second loop on a second side.

FIG. 10B shows a graft 1104 of the tendon with the staple 300 and the suture construct 1004 attached thereto. The graft 1104 can be passed through an interior of the sleeve 1002 (FIG. 10A) and then the sleeve 1002 (FIG. 10A) can be removed leaving the staple 300 and the suture construct 1004 in place and trapping the graft 1104. The closed loop 1006 and the tails 1008 can be tensioned in opposing directions to draw the wrapping of the suture construct 1004 tighter around the graft 1104 thereby trapping the graft 1104 and forcing the staple 300 against and into penetrating contact with the graft 1104.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The term “substantially”, “generally” or “about” mean within 15% of the value provided. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A system for connecting to a graft to in a reconstruction surgery, comprising:
a suture construct; and
a staple configured to anchor the suture construct to the graft.

2. The system of claim 1, wherein the suture construct includes a main portion, a link portion and a plurality of tails connected to and extending from the link portion, wherein the staple is configured to anchor the link portion to the graft.

3. The system of claim 2, wherein the suture construct includes a bridge with one or more features configured to receive portions of the plurality of tails.

4. The system of claim 2, wherein the staple includes one or more grooves configured to receive one or more of the plurality of tails therein.

5. The system of claim 4, wherein the one or more grooves are spaced longitudinally along a longitudinal length of the staple to receive the plurality of tails which are wrapped around the graft to form a cerclage.

6. The system of claim 2, wherein the staple includes one or more holes configured to receive one or more of the plurality of tails therein.

7. The system of claim 6, wherein the one or more holes are spaced longitudinally along a longitudinal length of the staple and the one or more holes are configured to receive one or more of the plurality of tails therein.

8. The system of claim 6, further comprising a needle coupled to a first end of the plurality of tails, wherein the first end opposes a second end that is coupled to or forms the link portion, wherein the one or more holes are each configured as a guide to pass the needle through the staple and into the link portion and the graft.

9. The system of claim 2, further comprising a card configured to carry the main portion of the suture construct.

10. The system of claim 1, further comprising an inserter configured to engage the staple and clamp the staple against the graft to cause penetration of the staple into the suture construct and the graft.

11. The system of claim 1, wherein the suture construct is configured to engage both a first side and a second side of the graft, wherein the staple is configured to anchor the suture construct to the first side of the graft, and further comprising a second staple configured to anchor the suture construct to the second side of the graft.

12. A method of connecting a suture construct to a graft in a reconstruction surgery, comprising:
harvesting the graft;
placing the suture construct adjacent the graft; and
anchoring the suture construct to the graft with a staple.

13. The method of claim 12, further comprising anchoring the suture construct to a second side of the graft with a second staple.

14. The method of claim 12, further comprising wrapping the suture construct around the graft and staple to form a cerclage.

15. The method of claim 14, further comprising passing the suture construct through one or more holes of the staple and piercing the suture construct captured by the staple and piercing the graft.

16. The method of claim 14, further comprising passing the suture construct through one or more grooves of the staple during the wrapping.

17. The method of claim 12, wherein anchoring the suture construct to the graft with the staple includes clamping the staple against the graft to cause penetration of the staple into the suture construct and the graft.

18. An assembly for a reconstruction surgery, comprising:
a suture construct including a main portion, a link portion and a plurality of tails connected to and extending from the link portion; and
a staple coupled to the link portion and configured to anchor the link portion to a graft, wherein when the link portion is anchored to the graft by the staple, the plurality of tails are configured to be wrapped around the graft and the staple to form a cerclage.

19. The assembly of claim 18, wherein the suture construct includes a bridge with one or more features comprising at least one of a groove and a hole configured to receive portions of the plurality of tails, further comprising a needle coupled to a first end of the plurality of tails, wherein the first end opposes a second end that is coupled to or forms the link portion, wherein the hole is configured as a guide to pass the needle through the staple and into the link portion and the graft.

20. The assembly of claim 18, further comprising:
a card carrying the main portion of the suture construct; and
an inserter configured to engage the staple and clamp the staple against the graft to cause penetration of the staple into the link portion and the graft;
wherein the assembly includes the anchor and a second anchor anchoring the suture construct to both a first side and a second side of the graft, respectively.
